SEQUENCE LISTING (9 sequences)

SEQ ID NO: 1 - DNA, 19 bp, synthetic construct
  source 1..19: mol_type = other DNA
  misc_difference 14: note = unmodified c, m5c or 5-hydroxymethylcytosine
ctcatgctgaaaacgtggt

SEQ ID NO: 2 - DNA, 18 bp, synthetic construct
  source 1..18: mol_type = other DNA
  misc_difference 10: note = unmodified c, m5c or 5-hydroxymethylcytosine
ggacaggaccagcatacg

SEQ ID NO: 3 - DNA, 26 bp, synthetic construct
  source 1..26: mol_type = other DNA
  misc_difference 23: note = unmodified c, m5c or 5-hydroxymethylcytosine
taactttgaccgtgagcagatgcgtc

SEQ ID NO: 4 - DNA, 26 bp, synthetic construct
  source 1..26: mol_type = other DNA
  misc_difference 22: note = unmodified c, m5c or 5-hydroxymethylcytosine
atcatcttcttcctcgtgcatcgagc

SEQ ID NO: 5 - DNA, 22 bp, synthetic construct
  source 1..22: mol_type = other DNA
ctcagagagaggctgatcacta

SEQ ID NO: 6 - DNA, 20 bp, synthetic construct
  source 1..20: mol_type = other DNA
gaacaacaacccgcaacatc

SEQ ID NO: 7 - DNA, 10 bp, synthetic construct
  source 1..10: mol_type = other DNA
  misc_difference 6: note = can be unmodified, or modified by one of these modifications: m5c, 5-hydroxymethylcytosine, beta-glucosyl-5-hydroxymethylcytosine or beta-6-azide-glucosyl-5-hydroxymethyl-cytosine
gaaaacgtgg

SEQ ID NO: 8 - DNA, 11 bp, synthetic construct
  source 1..11: mol_type = other DNA
  misc_difference 6: note = can be unmodified a or modified m6A or azide-modified adenine (N3-A)
gatcgaatatt

SEQ ID NO: 9 - skipped

(19) United States
(12) Patent Application Publication
(10) Pub. No.: US 2026/0028668 A1
(43) Pub. Date: Jan. 29, 2026

(54) DETECTION OF BASE MODIFICATIONS BY ENHANCING ELECTRICAL CONTRAST IN NANOPORES

(71) Applicant: Ramot at Tel-Aviv University Ltd., Tel-Aviv (IL)

(72) Inventors: Yuval EBENSTEIN, Tel-Aviv (IL); Yael MICHAELI-HOCH, Tel-Aviv (IL)

(73) Assignee: Ramot at Tel-Aviv University Ltd., Tel-Aviv (IL)

(21) Appl. No.: 19/099,809

(22) PCT Filed: Jul. 31, 2023
(86) PCT No.: PCT/IL2023/050792

§ 371 Date: Jan. 30, 2025

Related U.S. Application Data
(60) Provisional application No. 63/394,002, filed on Aug. 1, 2022.

(51) Int. Cl. C12Q 1/6869; C12Q 1/34; C12Q 1/48; C12Q 1/6809

(52) U.S. Cl. CPC C12Q 1/6869; C12Q 1/34; C12Q 1/48; C12Q 1/6809; C12Y 201/01037; C12Y 201/01072; C12Y 302/02009; C12Q 2600/154; C12Q 2600/156

(57) ABSTRACT

Provided herein is a method of detecting the presence or absence of a single naturally- or synthetically-modified nucleobase in a polynucleotide molecule, which is achieved by first contacting the polynucleotide molecule with one or more reagents capable of attaching a detectable moiety to at least one nucleobase of the polynucleotide molecule or to a nucleobase adjacent to the modified nucleobase, thereby forming a labeled nucleobase, while the presence or absence of the modified nucleobase is determined by the attachment. The polynucleotide molecule is then assayed using a nanopore device to detection the presence or absence of the labeled nucleobase, wherein the detectable moiety attached to at least one nucleobase in the polynucleotide molecule has a molecular weight that ranges from 40 to 1,000 Daltons, and the average pore diameter in the nanopore device is no more than 5 nanometers.

### RELATED APPLICATIONS

This application claims the benefit of priority of U.S. Provisional Patent Application No. 63/394,002 filed on 1 Aug. 2023, the contents of which are incorporated herein by reference in their entirety.

### SEQUENCE LISTING STATEMENT

The XML file, entitled “97066 Sequence Listing”, created on 26 Jul. 2023, comprising 12,288 bytes, submitted concurrently with the filing of this application is incorporated herein by reference.

### FIELD AND BACKGROUND OF THE INVENTION

The present invention, in some embodiments thereof, relates to polynucelotide assays, and more particularly, but not exclusively, to a novel methodology for identifying modified nucleotides.

DNA methylation is a biological process by which methyl groups are added to DNA. Cytosine can be methylated to form 5-methylcytosine (5mC) or N⁴-methycytosine (4mC), and adenine can be methylated to N⁶-methyladenine (m6A). In addition, 5mC can be oxidized to 5-hydroxymethylcytosine (5hmC), 5-formylcytosine (fC) or 5-carboxylcytosine (caC). In mammals, DNA methylation is almost exclusively found in CpG dinucleotides, in which the cytosines on both strands are usually methylated.

Nanopore sequencing, which was commercially released in 2014, allows the sequencing of a single molecule of DNA or RNA, without the need for PCR amplification. A bias voltage is applied across a membrane containing a nanopore in the presence of an electrolyte solution, such that a steady ionic current in the vicinity of the nanopore can be detected by electrodes near the membrane. A DNA/RNA molecule passing through a nanopore partially restricts the flow of ions, which is observed as an ionic current drop. Sequencing can be performed due to characteristic changes in the density of the electric current flowing through the nanopore.

Biological nanopore technology typically uses transmembrane proteins (porins) embedded in lipid membranes. Proteins can provide uniform pore structure and low translocation velocity (which facilitates measurements), but are relatively sensitive to environmental stress.

Solid state nanopore technology utilizes various metal or metal alloy substrates with nanometer-sized pores instead of protein nanopores. This allows for more stress tolerance, product longevity and ease of fabrication than obtained with proteins, but provides less uniformity and higher translocation velocity.

Various algorithmic techniques have been used to enhance nanopore sequencers, such as the “k-mer” approach, in which more than one base is analyzed at any one time, such that stretches of DNA are subject to repeat interrogation as the strand moves through the nanopore.

Algorithms have also been designed to detect modified nucleotides by nanopore sequencing: the Nanopolish software tool can detect 5mCG (5mC in a CpG site); signalAlign can detect 5mC, 5hmC and m6A; Tombo can detect 5mC and m6A; and mCaller, DeepSignal, DeepMod and Megalodon can detect m6A and 5mCG [Gouil & Keniry, Essays Biochem 2019, 63:639-648]. However, such approaches commonly suffer from a high false discovery rate and/or require appropriate training data [Amarasinghe et al., Genome Biol 2020, 21:30].

Gilboa et al. [ACS Nano 2016, 10:8861-8870] describes a method of quantifying unmethylated CpG dinucleotides and covalent coupling of DNA with synthetic cofactor analogs using DNA methyltransferases, followed by molecule-by-molecule electro-optical nanopore detection and quantification with single or multiple colors.

Sharim et al. [Genome Res 2019, 29:646-656] describes the use of M.TaqI (TaqI methyltransferase) to selectively transfer a fluorophore onto the adenine residue of TCGA recognition sites in which the CpG dinucleotide is unmethylated, followed by optical mapping uniformly stretched DNA fluorescently barcoded DNA molecules.

Michaeli et al. [Chem Commun (Camb) 2013, 49:8599-85601] describes specific labeling of 5hmc along genomic DNA molecules with a fluorescent reporter molecule, using introduction of azidoglucose by enzymatic glycosylation with T4 β-glucosyltransferase, followed by a click chemistry reaction.

Chen et al. [ACS Nano 2021, 15:2679-2685] describes high resolution nanopore DNA sequencing which utilizes labeling specific short sequence motifs with oligodeoxynucleotides using DNA methyltransferase.

Additional background art includes Johnson et al. [J Am Chem Soc 2017, 139:2750-2756]; Zhang et al. [Front Genet 2021, 12:672804]; and International Patent Application publications WO 2014/191981, WO 2017/081689, WO 2018/029693, WO 2019/234753 and WO 2021/250677.

### SUMMARY OF THE INVENTION

A method of detecting a presence or absence of a single naturally- or synthetically-modified nucleobase in a polynucleotide molecule, the method comprising (a) contacting said polynucleotide molecule with one or more reagents capable of attaching a detectable moiety to at least one nucleobase of said polynucleotide molecule, or to at least one nucleobase adjacent to at least one naturally-or synthetically-modified nucleobase in said polynucleotide molecule, to form a labeled nucleobase, wherein said attaching is determined by a presence or absence of said modified nucleobase; and (b) assaying said polynucleotide molecule using a nanopore device, thereby detecting a presence or absence of said labeled nucleobase of said polynucleotide molecule. A method of detecting a presence or absence of a single naturally-or synthetically-modified nucleobase in a polynucleotide molecule, the method comprising assaying a polynucleotide molecule using a nanopore device having an average pore diameter of no more than 5 nanometer, wherein at least one nucleobase in said polynucleotide molecule has a detectable having a molecular weight that ranges from 40 to 1,000 Daltons attached thereto, thereby detecting a presence or absence of said labeled nucleobase of said polynucleotide molecule.

According to an aspect of some embodiments of the invention there is provided a method of detecting a presence or absence of a single naturally-or synthetically-modified nucleobase in a polynucleotide molecule. The method is effected by

(a) contacting the polynucleotide molecule with one or more reagents capable of attaching a moiety to at least one nucleobase of the polynucleotide molecule to form a labeled nucleobase, wherein the attaching is determined by a presence or absence of the modified nucleobase; and

(b) assaying the polynucleotide molecule using a nanopore device,

thereby detecting a presence or absence of the labeled nucleobase of the polynucleotide molecule.

In some embodiments of the invention the nanopore device has an average pore diameter of no more than 5 nanometer.

In some embodiments of the invention the detectable moiety has a molecular weight of from 40 to 1,000 Daltons.

In some embodiments of the invention the one or more reagents include, without limitation, an enzyme, e.g., methyltransferase or methylase, such as, but not limited to, adenine methylase or CpG methylase, and/or DAM methyltransferase, Taq1 methyltransferase, Alu1 methyltransferase, BamH1 methyltransferase, CpG methyltransferase (M.SssI), CpG methyltransferase (M.MpeI), GpC methyltransferase (M.CviPI), EcoG2 methyltransferase, EcoRI methyltransferase, Hac3 methyltransferas, HhaI, Hpa2 methyltransferas and/or Msp1 methyltransferas optionally in combination with 5′-methylthioadenosine/S-adenosylhomocysteine nucleosidase (MTAN) enzyme.

According to an aspect of some embodiments of the invention there is provided a method of detecting a presence or absence of a single naturally-or synthetically-modified nucleobase in a polynucleotide molecule; the method is effected by assaying a polynucleotide molecule using a nanopore device having an average pore diameter of no more than 5 nanometer, wherein at least one nucleobase in the polynucleotide molecule has a detectable moiety having a molecular weight that ranges from 40 to 1,000 Daltons attached thereto, thereby detecting a presence or absence of the labeled nucleobase of the polynucleotide molecule.

In some embodiments of the invention the method further includes determining a sequence of the polynucleotide molecule by nanopore sequencing.

In some embodiments of the invention the nanopore device is a protein nanopore device.

In some embodiments of the invention the reagents are capable of selectively attaching the moiety to the modified nucleobase, and a presence of the labeled nucleobase is indicative of an initial presence of the modified nucleobase at the position of the labeled nucleobase.

In some embodiments of the invention the modified nucleobase is 5-hydroxymethylcytosine.

In some embodiments of the invention the reagents capable of selectively attaching the moiety to the modified nucleobase include, without limitation, β-glucosyltransferase and a uridine diphosphoglucose that includes a substituted or nonsubstituted glucose moiety.

In some embodiments of the invention a presence of the labeled nucleobase is indicative of an initial absence of the modified nucleobase at or near the position of the labeled nucleobase.

In some embodiments of the invention the reagents capable of selectively attaching the moiety to the modified nucleobase include a methyltransferase or methylase, such as adenine methylase or CpG methylase and/or in combination with MTAN enzyme.

In some embodiments of the invention the reagents capable of selectively attaching the moiety to the modified nucleobase further include an S-alkyl-S-adenosyl-homocysteine or synthetic analog thereof.

In some embodiments of the invention the modified nucleobase is 5-methylcytosine.

In some embodiments of the invention the method is for detecting an absence of 5-methylcytosine in a CpG dinucleotide.

In some embodiments of the invention the modified nucleobase is a modified adenine adjacent to the CpG dinucleotide.

In some embodiments of the invention the one or more reagents include DAM methyltransferase (DAM MTase), Taq1 methyltransferase, Alu1 methyltransferase, BamH1 methyltransferase, CpG methyltransferase (M.SssI), CpG methyltransferase (M.MpeI), GpC methyltransferase (M.CviPI), EcoG2 methyltransferase, EcoR1 methyltransferase, Hae3 methyltransferas, Hha1, Hpa2 methyltransferas, and/or Msp1 methyltransferas optionally in combination with 5′-methylthioadenosine/S-adenosylhomocysteine nucleosidase (MTAN) enzyme.

In some embodiments of the invention the method further includes analyzing data obtained from the nanopore device.

In some embodiments of the invention the analyzing data includes analyzing at least one parameter selected from the group consisting of shift in current, skipped events, unidentified k-mers, and modulation in dwell time.

In some embodiments of the invention a shift in current of the labeled nucleobase relative to a corresponding standard nucleobase is greater than a shift in current of the modified nucleobase relative to a corresponding standard nucleobase.

In some embodiments of the invention the difference between the shift in current of the labeled nucleobase and the shift in current of the non-labeled modified nucleobase is statistically significant.

In some embodiments of the invention, the mean shift in current of the labeled nucleobase is at least two standard deviations greater or smaller (different) than the mean of the shift in current of the non-labeled modified nucleobase.

In some embodiments of the invention, the resolution factor corresponding to the observed shift in current of the labeled nucleobase and the observed shift in current of the non-labeled modified nucleobase is at least 1.5.

In some embodiments, the shift in current of the labeled nucleobase is at least two-fold greater than the shift in current of the modified nucleobase.

As used herein the term “about” refers to ±10%. For example, the term “about 100 μm” encompasses the value 100 μm, as well as the values 90 μm, 91 μm, 92 μm, 93 μm, 94 μm, 95 μm, 96 μm, 97 μm, 98 μm, 98 μm, 99 μm, 100 μm, 101 μm, 102 μm, 103 μm, 104 μm, 105 μm, 106μm, 107 μm, 108 μm, 109 μm, and 110 μm.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the phrase “selected from the group consisting of” includes all members of the recited group, each member of the recited group, and all possible combinations. For example, selected from the group consisting of A, B, and C, includes A, only, as well as B, only, as well as C, only, as well as A and B, as well as A and C, as well as B and C. and as well as A, B, and C.

As used herein, the phrases “substantially devoid of” and/or “essentially devoid of” in the context of a certain substance, refer to a composition that is totally devoid of this substance or includes less than about 5, 1, 0.5 or 0.1 percent of the substance by total weight or volume of the composition. Alternatively, the phrases “substantially devoid of” and/or “essentially devoid of” in the context of a process, a method, a property or a characteristic, refer to a process, a composition, a structure or an article that is totally devoid of a certain process/method step, or a certain property or a certain characteristic, or a process/method wherein the certain process/method step is effected at less than about 5, 1, 0.5 or 0.1 percent compared to a given standard process/method, or property or a characteristic characterized by less than about 5, 1, 0.5 or 0.1 percent of the property or characteristic, compared to a given standard.

When applied to an original property, or a desired property, or an afforded property of an object or a composition, the term “substantially maintaining”, as used herein, means that the property has not change by more than 20%, 10% or more than 5% in the processed object or composition.

The term “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The words “optionally” or “alternatively” are used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3. from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the terms “process” and “method” refer to manners, means, techniques and procedures for accomplishing a given task including, but not limited to, those manners, means, techniques and procedures either known to, or readily developed from known manners, means, techniques and procedures by practitioners of the chemical, material, mechanical, computational and digital arts.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying figures. With specific reference now to the figures in detail, it is stressed that the particulars shown are by way of example and for purposes of illustrative discussion of embodiments of the invention. In this regard, the description taken with the figures makes apparent to those skilled in the art how embodiments of the invention may be practiced. In the drawings:

FIGS. 1A-B present histograms showing current level for cytosine (C), 5-methylcytosine (5mC), 5-hydroxymethylcytosine (5hmC) and 5-hydroxymethylcytosine tagged with glucose (5hmC-glu) or azidoglucose (5hmC-glu-azide) (FIG. 1A) and for adenine (A), methylated adenine (m6A) and azide-modified adenine (Azide-A) (FIG. 1B);

FIG. 2 presents a bar graph showing the effect of various cytosine modifications on four nanopore parameters (unidentified k-mers, shift from model current, dwell time, and skipped events);

FIG. 3 presents a genome browser view of an amplicon with different cytosine modifications (red arrow indicates the location of modified cytosine), identified by analysis of electrical signal and the abovementioned four nanopore parameters according to some embodiments of the invention (location of CpG sites also indicated); and

FIG. 4 presents a genome browser view of 5hmC peaks identified in mouse cortex in public data obtained by TAB-seq, in a control genomic DNA sample with native 5hmC, and in genomic DNA where 5hmC was modified to 5hmC-Glu-azide according to some embodiments of the invention (location of CpG sites also indicated).

### DESCRIPTION OF SPECIFIC EMBODIMENTS OF THE INVENTION

The present invention, in some embodiments thereof, relates to polynucelotide assays, and more particularly, but not exclusively, to a novel methodology for identifying modified nucleotides.

Before explaining at least one embodiment of the invention in detail, it is to be understood that the invention is not necessarily limited in its application to the details set forth in the following description or exemplified by the Examples. The disclosure is meant to encompass other embodiments or of being practiced or carried out in various ways.

The inventors have uncovered that the sensitivity of nanopore technology to modified nucleobases can be enhanced by chemical manipulations that selectively attach a chemical moiety to polynucleotides.

While reducing the present invention to practice, the inventors have shown that the current signal of 5hmc labeled with glucose or azidoglucose contrasts with the signal of cytosine (and 5mc) to a considerably greater degree than does the current signal of non-labeled 5hmc; and that the current signal of adenine labeled with an azide-substituted hydrocarbon contrasts with the signal of adenine to a considerably greater degree than does the current signal of m6A.

The inventors have further shown that embodiments of the invention can be applied using protein nanopore technology. The fundamental concept that is being employed in nanopore technology, such as employed in, for a non-limiting example, the Oxford Nanopore devices, of is pore-based sensing. This is a technique that uses a nanopore to measure the passage of molecules through nano-scale pores. A nanopore is a very small hole, typically only a few nanometers in diameter, that is embedded in a membrane. When a molecule passes through the nanopore, it causes a change in the electrical current that is flowing through the membrane. This change in current can be used to identify the molecule that passed through the nanopore.

In the case of protein nanopore technology, the nanopore is made of a protein; for example, alpha-hemolysin, which is a naturally occurring protein found in the cell walls of bacteria. When alpha-hemolysin, or a functionally similar protein, is embedded in a membrane, it forms a single-stranded alpha-helical channel that is about 2 nanometers wide. DNA and RNA molecules are also single-stranded, so they can pass through the alpha-hemolysin nanopore. As a DNA or RNA molecule passes through the nanopore, it causes a change in the electrical current that is flowing through the membrane. This change in current can be used to identify the sequence of nucleotides in the DNA or RNA molecule.

The present invention refers to nanopore technologies that allow DNA sequencing (detecting the label in its sequence context). While solid state nanopores have been used to detect bulky adducts, solid state (non-protein) nanopore technology cannot be used for sequencing, and it is limited to a qualitative “yes/no” per DNA fragment.

The present invention therefore provides a general approach of detecting modifications in a polynucleotide, even detection of a single modified nucleotide in a polynucleotide, based on the ability to selectively attach a detectable moiety only to the modified nucleotide of interest. The attachment of the detectable moiety (e.g., a bulky moiety, a fluorescent moicty, a chromophore, a charged moiety, and/or a radioactive isotope) changes one or more properties of the polynucleotide, which increases a nanopore-detectable signal, such that the presence of the modified nucleotide of interest in the polynucleotide can be determined.

The capacity of a reagent to generally further modify nucleobases, modified or not, is not sufficient to achieve the objectives of the present invention, hence, the suitable reagent is capable of attaching a moicty to a specific modified nucleobase(s), and by selective it is meant that nucleobases that do not correspond to the specific modified nucleobase will not be labeled with a detectable moiety. Some labeling reagents that require to exhibit target specificity are enzymes, as in the case of the present invention, are enzymes, such as, but not limited to methyltransferase or methylase.

The invention can overcome the present difficulty in identifying naturally occurring polynucleotide modifications using nanopore technology, due to the very low contrast between various modified nucleobases and the corresponding non-modified nucleobase, upon being passed through a nanopore. As a result, features of nanopore technology which are advantageous in studying polynucleotide modification may be exploited, such as the ability to avoid PCR amplification, a process during which nucleotide modifications are commonly lost.

Embodiments of the invention can be useful in facilitating epigenome research, for example, by providing accurate and simultaneous long-range epigenetic mapping of multiple epigenetic marks, based on the existing commercial technology of nanopore sequencing.

Furthermore, as the effect of the attached chemical moiety on nanopore signals is largely due to bulkiness—e.g., as opposed to optical absorption or fluorescence—a very wide variety of chemical moieties may be utilized, which may be useful in reducing costs and/or enhancing product stability.

The present invention provides a method for detecting the presence or absence of one or more naturally-or synthetically-modified nucleobases in a polynucleotide molecule. The method involves contacting the polynucleotide molecule with one or more reagents capable of selectively attaching a detectable moiety (a label) to the modified nucleobase, and assaying the polynucleotide molecule using a nanopore device, and the nanopore device may be a protein nanopore device.

The nanopore technology referred to herein as “protein nanopore device” is sometimes referred to as “single-molecule DNA sequencing”, which is a method of DNA sequencing that allows the sequence of a DNA molecule to be determined one molecule at a time. In single-molecule DNA sequencing, a DNA molecule is passed through a nanopore, which is a tiny hole in a membrane, formed by proteins embedded in the membrane. As the DNA molecule passes through the nanopore, it blocks the flow of charged species (current), and the amount of current that is blocked depends on the sequence of the DNA molecule. By measuring the amount of current that is blocked, the sequence of the DNA molecule can be determined. The nanopore technology that is used in single-molecule DNA sequencing is the pore-forming protein, wherein the pore-forming proteins are proteins that form pores in membranes. The pores that are formed by pore-forming proteins are typically very small, which makes them ideal for single-molecule DNA sequencing. There are a number of different pore-forming proteins that can be used for single-molecule DNA sequencing, and some of the most commonly used pore-forming proteins include a-hemolysin, MspA, CsgG and engineered variants thereof.

In some embodiments of the present invention, the use of a nanopore device includes the use of a helicase to restrict the translocation velocity (a.k.a., translocation speed). Helicase is a protein that unwinds DNA, which is conducive to sequencing. The rate at which the helicase can move along a DNA molecule is limited by the translocation velocity of the DNA molecule through the nanopore, whereas the translocation velocity is the speed at which the DNA molecule moves through the nanopore. The translocation velocity is determined by a number of factors, including the size of the nanopore, the size of the DNA molecule, the charge of the DNA molecule, and the viscosity of the solution, whereas the translocation velocity is typically on the order of a few nucleotides per second. The presence of helicase can restrict the translocation velocity in a nanopore device: helicase can move along the DNA molecule at a slower speed than the DNA molecule can move through the nanopore; or, helicase can cause the DNA molecule to coil up, which can make it more difficult for the DNA molecule to move through the nanopore. There are a number of techniques that can be used to affect the restriction of translocation velocity by helicase; for example, the helicase can be modified to make it more or less active or to bind to the DNA molecule more or less tightly.

The presence or absence of the labeled nucleobase is indicative of the initial presence or absence of the modified nucleobase. The nanopore device may have an average pore diameter of no more than 5 nanometers, and the detectable moicty may have a molecular weight ranging from 40 to 1,000 Daltons. The reagents capable of selectively attaching a detectable moiety to a specific modified nucleobase, or to a nucleobase adjacent to a modified nucleobase, may comprise an enzyme such as a methyltransferase or methylase. The method may also include determining the sequence of the polynucleotide molecule by nanopore sequencing.

Thus, according to an aspect of some embodiments of the invention, there is provided a method of detecting a presence or absence of a single naturally-or synthetically-modified nucleobase in a polynucleotide molecule, which is effected by

(a) contacting the polynucleotide molecule with one or more reagents capable of attaching a moiety to at least one nucleobase of the polynucleotide molecule to form a labeled nucleobase in the polynucleotide, wherein the attachment is determined by detection of the presence or absence of the modified nucleobase in the polynucleotide; and

(b) assaying the polynucleotide molecule using a nanopore device, thereby detecting a presence or absence of said labeled nucleobase of said polynucleotide molecule.

As known in the art, as well as in the context of the present invention, the term “nucleobase” refers to nitrogenous base that is one of the building blocks of DNA and RNA. The term “polynucleotide molecule” refers to a chain of nucleotides, such as, for example DNA or RNA. The term “modified nucleobase” refers to a nucleobase that has been chemically changed. The term “label” refers to a molecule that can be attached to another molecule to render it easier to detect.

The phrase “labeling agent” refers to an agent capable of attaching to a target molecule (e.g., a polynucleotide molecule, a DNA molecule) so as to form a readily detectable derivative of the target molecule. The labeling agent typically comprises a detectable moiety (e.g., a bulky moiety, a fluorescent moiety, a chromophore, a charged moiety, and/or a radioactive isotope) or a detectable moiety is formed upon attachment of the labeling agent to the target molecule.

In the context of the present invention, the term “moiety” refers to a specific group of atoms within a molecule, or a distinct and identifiable part, fragment or functional group within that molecule, that is responsible for a characteristic property, function, activity, reactivity or a chemical reaction of that molecule. In some embodiments, the terms “moiety” and “functional group” are interchanged. Generally, a moiety is given the name of its precursor, another molecule that is almost identical to the moiety, except for not being attached to another molecule. For example, is a nucleobase is labeled with a moiety derived from glucose, the moiety is named or referred to as a glucose moicty.

According to some embodiments, the detectable moiety is covalently attached to the DNA molecule. In some embodiments, the detectable moiety is a covalently attached moicty having a molecular weight that ranges 40-1,000 Daltons. Non-limiting examples of detectable moieties, according to some embodiments of the present invention, include, without limitation, C2-6 alkanes (e.g., ethyl), C2-6 alkenes (e.g., ethylene), C2-6 alkynes (e.g., propargyl), carboxyl, glucose, biotin, azide, cyclooctyne, triazole, maleimide, and fluorophores such as fluorescein, rhodamine, cyanine, BODIPY and quantum dots.

Attaching a labeling agent to a DNA molecule (according to any of the respective embodiments described herein) may optionally be effected using suitable reagents, such as are known in the art. For example, WO 2014/191981 describes a method of labeling 5-hmC along a DNA molecule, by attaching a 5-hmC-specific labeling agent to the DNA and extending the DNA molecule. UDP-6-N3-glucose may be used as a reagent modifying 5-hmC with an azide group, which can be further labeled using click chemistry.

In some embodiments of any of the embodiments described herein, relating to a detectable moiety imparted to the polynucleotide molecule by the labeling agent, according to any of the respective embodiments described herein, is a fluorescent detectable moicty. In some embodiments, determining the presence of the detectable moiety in the polynucleotide molecule, according to any of the respective embodiments described herein, is effected by detecting the fluorescence corresponding to the detectable moiety in the polynucleotide.

In some embodiments of any of the embodiments described herein, relating to a detectable moiety imparted to the polynucleotide molecule by the labeling agent, according to any of the respective embodiments described herein, is a bulky detectable moiety. In some embodiments, determining the presence of the detectable moiety in the polynucleotide molecule, according to any of the respective embodiments described herein, is effected by detecting bulkiness (a change in size) corresponding to the detectable moiety in the polynucleotide.

In some embodiments of any of the embodiments described herein, relating to a detectable moicty imparted to the polynucleotide molecule by the labeling agent, according to any of the respective embodiments described herein, is a chromophore detectable moicty. In some embodiments, determining the presence of the detectable moiety in the polynucleotide molecule, according to any of the respective embodiments described herein, is effected by detecting the chromophore corresponding to the detectable moiety in the polynucleotide.

In some embodiments of any of the embodiments described herein, relating to a detectable moicty imparted to the polynucleotide molecule by the labeling agent, according to any of the respective embodiments described herein, is a charged detectable moiety. In some embodiments, determining the presence of the detectable moiety in the polynucleotide molecule, according to any of the respective embodiments described herein, is effected by detecting the charge corresponding to the detectable moiety in the polynucleotide.

In some embodiments of any of the embodiments described herein, relating to a detectable moiety imparted to the polynucleotide molecule by the labeling agent, according to any of the respective embodiments described herein, is a detectable moicty comprising a radioactive isotope. In some embodiments, determining the presence of the detectable moicty in the polynucleotide molecule, according to any of the respective embodiments described herein, is effected by detecting the radioactive isotope corresponding to the detectable moiety in the polynucleotide.

The term “nanopore device” refers to a device and a methodology that uses nano-scale holes embedded in a thin membrane structure to detect minute electric potential variations when charged molecules smaller than the nanopore pass through the hole. This technology allows for rapid, real-time sequencing of DNA or RNA molecules. In the context of the method described above, a nanopore device is used to assay the polynucleotide molecule and detect the presence or absence of the labeled nucleobase present in the DNA or RNA molecule. In the context of the above, a modified nucleobase can be labeled with a moiety that will cause a larger change in the electrical current when it passes through the nanopore. This allows the modified nucleobase to be detected more unambiguously, thereby enabling the identification of a modified nucleobase in a polynucleotide molecule with greater accuracy and sensitivity.

According to some embodiments of the invention the reagents capable of selectively attaching a detectable moiety to a modified nucleobase, or to a nucleobase adjacent to a modified nucleobase, comprise an enzyme, e.g., methyltransferase or methylase. Methyltransferase or methylase are enzymes that catalyze the transfer of a methyl group from a donor molecule to an acceptor molecule. In the context of some embodiments of the present invention, these enzymes are DNA or RNA methyltransferases, which means they transfer a methyl group to a nucleobase in a DNA or RNA molecule. This process is known as methylation and can have various effects on the function and expression of genes. Some of these enzymes, such as CpG methylase and M.SssI, specifically target CpG sites in DNA, while others, such as GpC methyltransferase (M.CviPI), target different nucleotide sequences. MTAN enzyme is a type of nucleosidase that catalyzes the hydrolysis of 5′-methylthioadenosine (MTA) to adenine and 5-methylthioribose-1-phosphate.

Methyltransferase or methylase, suitable for use as reagents capable of attaching a moicty to a modified nucleobase selectively include, but are not limited to, adenine methylase or CpG methylase, and/or DAM methyltransferase, Taq1 methyltransferase, Alu1 methyltransferase, BamH1 methyltransferase, CpG methyltransferase (M.SssI), CpG methyltransferase (M.MpeI), GpC methyltransferase (M.CviPI), EcoG2 methyltransferase, EcoRI methyltransferase, Hac3 methyltransferas, HhaI, Hpa2 methyltransferas and/or Msp1 methyltransferas optionally in combination with MTAN enzyme.

In some embodiments, the herein-mentioned enzymes can be used in combination with other enzymes to modify DNA in a specific way. For example, TET enzymes (a family of ten-eleven translocation methylcytosine dioxygenases) can be used to convert 5-methylcytosine to 5-hydroxymethylcytosine, which is a different type of DNA methylation mark. Additional methyltransferases, their catalytic activity and use thereof to selectively label modified nucleobases, are described, for example, in U.S. Pat. No. 8,008,007, which is incorporated by reference as if fully set forth here.

According to an aspect of some embodiments of the invention there is provided a method of detecting a presence or absence of a single naturally-or synthetically-modified nucleobase in a polynucleotide molecule. The method comprising assaying a polynucleotide molecule using a nanopore device having an average pore diameter of no more than 5 nanometer, according to some embodiments, wherein at least one nucleobase in the polynucleotide molecule has a detectable moiety having a molecular weight that ranges from 40 to 1,000 Daltons attached thereto, according to some embodiments, thereby detecting a presence or absence of the labeled nucleobase of the polynucleotide molecule.

According to some embodiments of the invention the detectable moiety has a molecular weight of from 40 to 1,000 Daltons. In some embodiments, the detectable moiety has a molecular weight of 40-500 Da, 200-800 Da, and 400-1,000 Da, and any sub-ranges thereof.

In some embodiments of the invention the nanopore device has an average pore diameter of no more than 2 nm, 3 nm, 4 nm or 5 nanometer. In some embodiments, the average pore diameter ranges 2-3 nm, 2-4 or 2-5 nm.

According to some embodiments of the invention the method further comprises determining a sequence of the polynucleotide molecule by nanopore sequencing.

According to some embodiments of the invention the nanopore device is a protein nanopore device.

According to some embodiments of the invention the one or more reagents are capable of selectively attaching the detectable moiety to the modified nucleobase, and a presence of the labeled nucleobase is indicative of an initial presence of the modified nucleobase at the position of the labeled nucleobase. The position of the labeled nucleobase in the polynucleotide can be determined upon sequencing the polynucleotide and monitoring the presence of the labeled nucleobase in the process.

According to some embodiments of the invention the modified nucleobase is 5-hydroxymethylcytosine.

According to some embodiments of the invention the one or more reagents comprise β-glucosyltransferase and a uridine diphosphoglucose comprising a substituted or non-substituted glucose moiety.

According to some embodiments of the invention a presence of the labeled nucleobase is indicative of an initial absence of the (non-labeled) modified nucleobase at or near the position of the labeled nucleobase. For example, Mtaq.I labels the adenine in the motif TCGA, and it will only label the Adenin if the close-by cytosine is unmodified. In another example, a DNA damage adduct may be enzymatically excised and the proximal bases will be labeled by modified nucleotides by a polymerase.

According to some embodiments of the invention the one or more reagents comprise a methyltransferase or methylase, such as adenine methylase or CpG methylase and/or in combination with MTAN enzyme.

According to some embodiments of the invention the one or more reagents further comprise an S-alkyl-S-adenosyl-homocysteine or synthetic analog thereof.

It is noted herein that chromatin accessibility may play a factor in the presently provided methods, e.g., in cases of DNA modifying enzymes with a high contrast cofactor that may be applied to permeabilized nuclei and label only the accessible DNA; once the DNA is stripped from protein and sequenced, only open chromatin regions will be marked by the label.

Alternatively, the labeling enzyme may be fused to a specific antibody against a DNA binder such as a transcription factor; after binding to the transcription factor, the enzyme tethered to the antibody will label the DNA proximal to the binder.

Further alternatively, the labeling enzyme is bound to G or A protein that will bind specifically to antibodies; first an antibody is applied to the nuclei, and in a second step the labeling enzyme is introduced, binds to the antibody and marks the DNA external to the site.

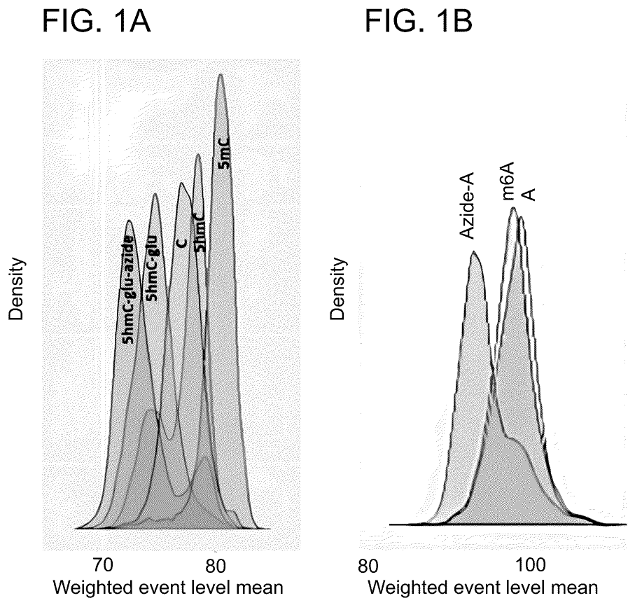

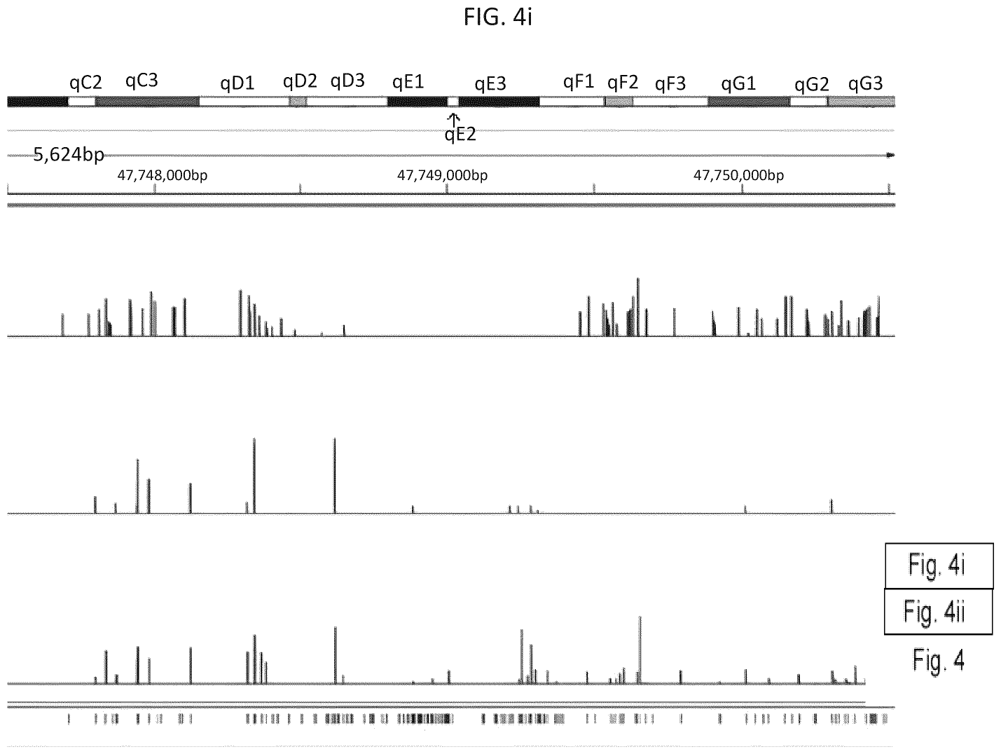

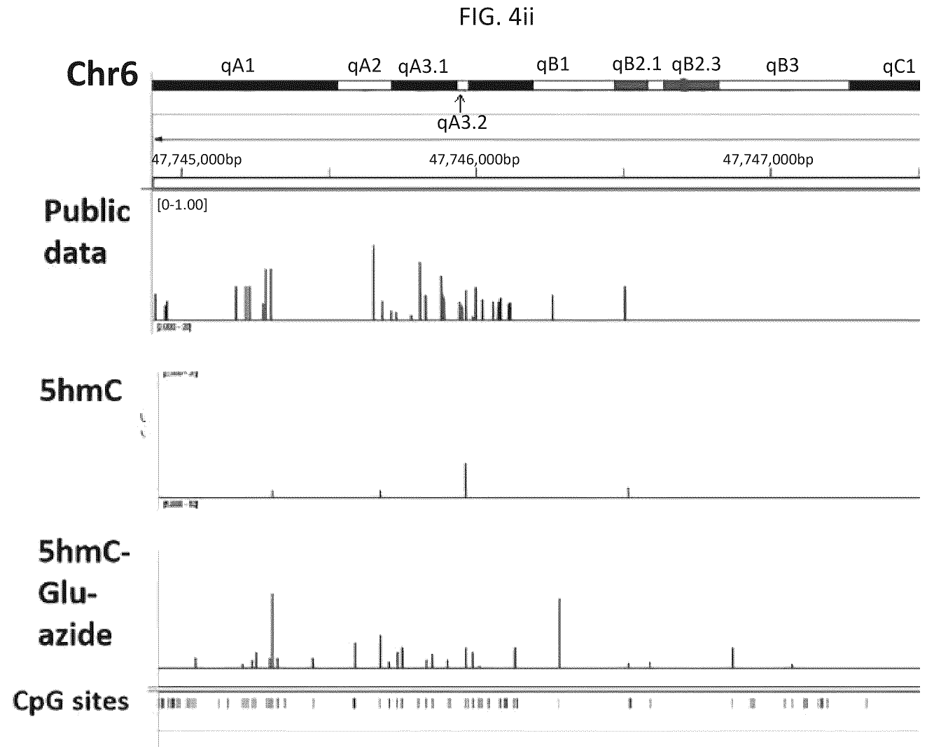

These concepts were demonstrated in the Examples section, using methylation enzymes and regular methylation cofactors (adomet/SAM) that produced a relatively low signal. For example, when using adenine methylaze like DAM or hia5, the resulting signal is poor, however when using an azide to label adenine the signal is high (see, FIGS. 1A-B and FIG. 4).

According to some embodiments, the modified nucleobase is an isomer of methylcytosine or methyladenine, such as, without limitation, 5-methylcytosine, N6-methylcytosine, 5-hydroxymethylcytosine, 5-formylcytosine, 5-methyladenine, 6-methyladenine and N1-methyladeninc.

According to some embodiments of the invention the modified nucleobase is a methylcytosine, such as 5-methylcytosinc.

According to some embodiments of the invention the method is for detecting an absence of 5-methylcytosine in a CpG dinucleotide.

According to some embodiments of the invention the modified nucleobase is a modified adenine adjacent to the CpG dinucleotide.

It is to be understood that the methodologies provided in the present disclosure are not limited to the labeling agents mentioned herein, and can be applied using many other labeling agents in the form of enzymes, such as list of methyltransferases. The database REBASE [Roberts, R. J. et al., “REBASE: restriction enzymes and methyltransferases”, Nucleic acids research, 2003, 31 (1), pp 418-20] provides comprehensive information about restriction enzymes, DNA methyltransferases and related proteins such as nicking enzymes, specificity subunits and control proteins. REBASE contains recognition and cleavage sites, isoschizomers, commercial availability, crystal and sequence data. Homing endonucleases are also included. REBASE contains the most complete and up-to-date information about the methylation sensitivity of restriction endonucleases. The data is available on the web (rebasc (dot) neb (dot) com), and the list of methylases is incorporated herein by reference.

An exemplary methylase is DNMT1, which is a DNA methyltransferase that is responsible for maintaining methylation patterns in DNA. It is similar to Hia5 in that it can methylate a wide variety of DNA sequences; however, DNMT1 is more specific than Hia5, and it is most active at CpG sequences.

Another exemplary methylase is DNMT3A (and DNMT3B), which is a DNA methyltransferase that is responsible for de novo methylation of DNA. It is similar to Hia5 in that it is a promiscuous methyltransferase; however, DNMT3A is more active at cytosine residues that are not followed by guanine (CpG) sequences. According to some embodiments of the invention the one or more reagents comprise DAM methyltransferase, Taq1 methyltransferase, Alu1 methyltransferase, BamH1 methyltransferase, CpG methyltransferase (M.SssI), CpG methyltransferase (M.MpeI), GpC methyltransferase (M.CviPI), EcoG2 methyltransferase, EcoRI methyltransferase, Hae3 methyltransferas, Hha1, Hpa2 methyltransferas, DNA (cytosine-5-)-methyltransferase 1 (DNMT1; EC 2.1.1.37), DNA methyltransferase 3A (DNMT3A; EC 2.1.1.38), DNA methyltransferase 3B (DNMT3B; EC 2.1.1.39), DAM MTase and/or Msp1 methyltransferas optionally in combination with 5′-methylthioadenosine/S-adenosylhomocysteine nucleosidase (MTAN) enzyme.

Methylation and hydroxymethylation are two naturally occurring modifications of DNA. Methylation occurs when a methyl group (CH₃) is added to a nucleobase in DNA, while hydroxymethylation occurs when a methyl group is oxidized or the full modification is added to DNA. Both methylation and hydroxymethylation are involved in regulating gene expression. Methylation and hydroxymethylation can be used to detect other genomic information, such as

DNA damage or the presence of genetic mutations. However, there are two challenges to using methylation and hydroxymethylation for this purpose. The first challenge is that methylation and hydroxymethylation are naturally occurring modifications, which means that there is always some level of background methylation and hydroxymethylation in DNA, even in healthy cells. This background methylation and hydroxymethylation can make it difficult to detect changes in methylation or hydroxymethylation that are caused by other factors, such as DNA damage or genetic mutations. The second challenge is that methylation and hydroxymethylation are not well discriminated, which means that it can be difficult to distinguish between different types of methylation and hydroxymethylation, even using sophisticated analytical techniques. This can make it difficult to track changes in methylation or hydroxymethylation over time or to compare methylation or hydroxymethylation levels between different cell types. Same stands for methylation of Adenine for labeling. Modified adducts can be used to overcome the challenges of using methylation and hydroxymethylation for detecting other genomic information. Modified adducts are molecules that are attached to DNA after it has been modified.

By using modified adducts, we can create a unique signature for each type of natural genomic modification. This allows us to multiplex different types of observables simultaneously, either by co-detection of natural and synthetic adducts or by detection of a combination of different synthetic adducts. For example, one could use a methyl adduct to detect methylation, a hydroxymethyl adduct to detect hydroxymethylation, and a third type of adduct to detect a different type of genomic modification. This would allow tracking multiple types of genomic modifications in a single experiment. According to some embodiments of the invention, the method further comprises analyzing data obtained from the nanopore device. According to some embodiments of the invention, analyzing data comprises detecting and analyzing nanopore-detectable events (signals) such as, without limitation, a shift in current, skipped events, unidentified k-mers, and modulation in dwell time, which also constitute some of the detectable causes of potential artifacts that can occur during nanopore sequencing.

DNA or RNA modification: the DNA or RNA molecule may be modified such that it does not pass through the nanopore smoothly, which can cause a shift in current.

k-Mers are short sequences of nucleotides that are used to identify DNA or RNA molecules. If the nanopore misreads a k-mer, it may identify the molecule as a different molecule, hence, labeling a modified nucleobase can be seen as an unidentified k-mer. Since the available nanopores have a predetermined width, there are several bases in the pore at any given time, all effecting the recorded current, and they are defined as k-mers, with k equals the number of bases affecting current.

The dwell time is the amount of time that the DNA or RNA molecule spends in the nanopore, therefore a modulated dwell time can be can be taken as detection of a labeled modified nucleobase.

Since these variations can be difficult to detect and identify, there are a number of techniques that can be used to increase the sensitivity of the device, which include:

Data filtering: Data filtering can be used to remove events that are likely to be artifacts. For example, events with large shifts in current or skipped events can be filtered out.

Error correction: Error correction algorithms can be used to correct errors in the sequence. These algorithms use statistical methods to identify and correct errors.

Quality assessment: Quality assessment tools can be used to assess the quality of the sequence data. These tools can identify events that are likely to be artifacts and provide information about the accuracy of the sequence.

By using these techniques, it is possible to reduce the impact of artifacts on a nanopore sequencing data.

According to some embodiments of the invention, a shift in current of the labeled nucleobase relative to a corresponding standard nucleobase is greater than a shift in current of the modified nucleobase relative to a corresponding standard nucleobase. In practice, for each nanopore device run, current shift measurement results are collected, processed and analyzed as a statistical event using multiple individual data points, whereas the results are typically provided as a weighted event level mean. There are a few parameters that can be used to resolve two almost overlapping events from one another, seen as two partially overlapping bell curves: mean, standard deviation, and overlap. The value that is used to signify that two events are distinguishable is typically a threshold value, which is the minimum amount of overlap that is required for the two curves to be considered distinguishable. The threshold value is typically chosen based on the application. For example, if the two events represent the distribution of two different molecular entities, the threshold value might be chosen to be the point at which the probability of misidentification is equal to 0.05. This means that there is a 5% chance of misidentification of one molecular entity with another. Alternatively, a threshold value is a significant difference equal or greater than 0.5 standard deviations, which means that the means of the two events are different by at least half of a standard deviation.

For example, for a shift in current to be seen as significant and corresponding to the presence of a labeled modified nucleobase in the polynucleotide, the change in the current shift should be at least statistically significant. In the context of resolution between two peaks, as in the case of analyzing and comparing the effect of the label on the shift in current (see, e.g., FIGS. 1A-B), statistical significance is typically measured using the resolution factor, which is the ratio of the peak widths to the distance between the peaks. A resolution factor of 1.5 is often considered to be the minimum required for two peaks to be considered resolved. This means that the peaks are at least 1.5 times wider than the distance between them. However, the choice of a resolution factor cutoff is somewhat arbitrary, and it is important to consider the context of the study when interpreting the results. For example, a resolution factor of 1.5 may be considered sufficient for a study that is trying to identify two different compounds, but it may not be sufficient for a study that is trying to quantify the amount of each compound.

A higher resolution factor indicates that the peaks are more clearly separated. A resolution factor of 2 between the peak corresponding to a shift in current of the non-labeled modified nucleobase and the peak corresponding to the shift in current of the labeled modified nucleobase, or higher, is preferred in some embodiments of the present invention.

In the context of resolving two peaks (two bell-curves corresponding to two sets of observations, wherein the observation is measurement of a shift in current), statistically significant is typically considered to be a difference in means that is greater than or equal to 1.96 standard deviations. This is based on the 68-95-99.7 rule, which states that 68% of the data in a normal distribution will fall within 1 standard deviation of the mean, 95% of the data will fall within 2standard deviations of the mean, and 99.7% of the data will fall within 3 standard deviations of the mean. Hence, if the mean of the two peaks (bell-curves) is different by 1.96 standard deviations, then there is a 95% chance that the difference is real and not due to chance.

In the context of the present invention, the practical significance of a difference in means will depend on the parameters and results of the study. For example, if the shift in current is very small, then a difference in means of 1.96 standard deviations may not be practically significant. However, if the shift in current is large, then a difference in means of 1.96 standard deviations may be practically significant.

Hence, according to some embodiments of the invention, the mean shift in current of the labeled nucleobase is at least two standard deviations greater or smaller (different) than the mean of the shift in current of the non-labeled modified nucleobase.

Unless otherwise defined, all technical and/or scientific terms used herein have the same meaning as commonly understood by one of ordinary skill in the art to which the invention pertains.

Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### MATERIALS AND METHOD

### Materials:

Ado YnAzide was synthesized inhouse.

β-Glucosyl-transferase (from T4 bacteriophages) was obtained from New England Biolabs.

MyTaq™ Red Mix was obtained from Bioline.

NEBuffer™ 3 was obtained from New England Biolabs.

Proteinase K was obtained from Sigma-Aldrich.

M.TaqI (TaqI methyltransferase), with 10x CutSmart® buffer and S-adenosyl-L-methionine, was obtained from New England Biolabs.

UDP-glucose was obtained from New England Biolabs.

UDP-6-N3-Glucose was synthesized in house.

### Preparation of DNA fragments with modified nucleotides:

Synthetic DNA was generated by amplification of lambda DNA to yield 1 kb amplicons (positions 10003, 39608, and 43,640).

In order to generate DNA with modified cytosine, primers containing modified cytosine at a specific position were used. Likewise, control DNA without modified cytosine was prepared using primers with unmodified cytosine at that position. The following primers were used for fragments #1 and #2, wherein the underlined cytosines were either unmodified cytosine (C), 5-methylcytosine (5mC) or 5-hydroxymethylcytosine (5hmC):

### Fragment #1 (10,003-11054):

Position 10,003 forward-
(SEQ ID No. 1)
CTCATGCTGAAAACGTGGT
Position 11,054 reverse-
(SEQ ID NO. 2)
GGACAGGACCAGCATACG

### Fragment #2 (39,608-42,407):

Position 39,608 forward-
(SEQ ID No. 3)
TAACTTTGACCGTGAGCAGATGCGTC
Position 42,407 reverse-
(SEQ ID NO. 4)
ATCATCTTCTTCCTCGTGCATCGAGC

For PCR amplification, 100 ng of lambda genomic DNA was mixed with 25 μL of MyTaq™ Red Mix, 2 μL of each primer (10 μM stock concentration), and ultra-pure water to a volume of 50 μL. Each fragment was amplified using either the unmodified primer, the 5mC-containing primer or the 5hmC-containing primer.

The mixture was placed in a thermocycler for the following program:

| Step | Temperature | Time | | Cycles |
|---|---|---|---|---|
| Initial denaturation | 95° C. | 1 | minute | 1 |
| Denaturation | 95° C. | 15 | seconds | |
| Annealing | 58° C. | 15 | seconds | 30 |
| Extension | 72° C. | 3 | minutes | |
| Final extension | 72° C. | 5 | minutes | 1 |

In order to generate DNA with modified or unmodified adenine, the lambda DNA was amplified using the following primers:

Position 46,340 Forward-
(SEQ ID No. 5)
CTCAGAGAGAGGCTGATCACTA
Position 48,266 Reverse-
(SEQ ID NO. 6)
GAACAACAACCCGCAACATC

For PCR amplification, 100 ng of lambda genomic DNA was mixed with 25 μL of MyTaq™ Red Mix, 2 μL of each primer (10 μM stock concentration), and ultra-pure water to a volume of 50 μL.

The mixture was placed in a thermocycler for the following program:

| Step | Temperature | Time | | Cycles |
|---|---|---|---|---|
| Initial denaturation | 95° C. | 1 | minute | 1 |
| Denaturation | 95° C. | 15 | seconds | |
| Annealing | 58° C. | 15 | seconds | 30 |
| Extension | 72° C. | 3 | minutes | |
| Final extension | 72° C. | 5 | minutes | 1 |

Following amplification, all of the DNA fragments were purified using a QIAquick™ PCR purification kit (Qiagen).

### Nanopore Libraries:

Nanopore libraries were constructed using a ligation sequencing kit (Oxford Nanopore Technologies, cat. #SQK-LSK-109) in combination with a barcoding kit (Oxford Nanopore Technologies, cat. #EXP-NBD-104). The libraries were then loaded on an R9.4.1 nanopore flowcell (Oxford Nanopore Technologies, cat. #MIN-FLO-106D).

For studies of cytosine modification, five samples were combined in a single run: unmodified cytosine, 5mC, 5hmC, 5hmC-Glu (5-gmC), and 5hmC-Glu-azide (N3-5-gmC). Each sample contained a mixture of two fragments, representing positions 10,003-11054 and 39,608-42,407.

For studies of adenine modification, three samples were combined in a single run: unmodified adenine, methylated adenine (m6A), and azide-modified adenine (N3-A).

### Example 1

### Detection of Modified Nucleotides using Nanopore Sequencing

Modified cytosine was labeled with bulky groups in order to evaluate whether this facilitates identification by nanopore sequencer.

In order to investigate cytosine modification, synthetic DNA (in the form of 1 kb amplicons) was generated using primers that contain either unmodified cytosine (C), 5-methylcytosine (5mC) or 5-hydroxymethylcytosine (5hmC) at a predetermined position, as described in the Materials and Methods section hereinabove. A portion of the 5hmC-containing amplicons were then further modified by attaching a glucose or 6-azidoglucose moiety to the hydroxy group of 5hmC (to form β-glucosyl-5-hydroxymethylcytosine or 6-azido-β-glucosyl-5-hydroxymethylcytosine, respectively), using T4 β-glucosyltransferase (β-GT) in the presence of uridine diphosphoglucose (UDP-Glu) or its azide derivative (UDP-6-N3-Glu), according to procedures such as described by Michaeli et al. [Chem Commun (Camb) 2013, 49:8599-85601].

Specifically, 1 μg amplified DNA was incubated with 3 μL NEBuffer™ 3, 3 μL T4 β-glucosyl-transferase, 200 μM of cofactor (either UDP-Glu or UDP-6-N3-Glu), and ultra-pure water to a final volume of 30 μL. The DNA was incubated overnight at 37° C. and then purified using a QIAquick™ PCR purification kit (Qiagen).

In order to investigate adenine modification, M.TaqI (TaqI methyltransferase) was used according to procedures such as described in Sharim et al. [Genome Res 2019, 29:646-656]. Methyltransferases can use synthetic cofactor analogs to incorporate other chemical moieties besides simple methyl groups onto bases. The amplified fragment contained three sites of the M.TaqI recognition sequence TCGA.

Specifically, 1.5 μg amplified and purified DNA was incubated with 2.25 L of M.TaqI, 7.5 μL of CutSmart® buffer, 80 μM (final concentration) of either the native cofactor AdoMet (S-adenosyl-L-methionine) or its azide-containing analog AdoYnAzide, and ultra-pure water to a final volume of 75 μL. AdoMet is suitable for transfer of a methyl group to adenine, whereas AdoYnAzide is suitable for transfer of a bulkier 6-azido-hex-2-yn-yl group. The reaction was incubated for 1 hour at 60° C., followed by addition of 40 mg of proteinase K for 2 hours at 45° C. The DNA was then purified with QIAquick™ PCR purification kit.

The current distribution signals associated with cytosine, 5-methylcytosine, 5-hydroxymethylcytosine, β-glucosyl-5-hydroxymethylcytosine (5gmC) and β-6-azide-glucosyl-5-hydroxymethyl-cytosine (N₃-5gmC) in the sequence GAAAACGTGG (SEQ ID No. 7) were compared. Upon examination of the yield from these amplicons, no significant changes were observed which could be attributed to the modifications, with 35,832 reads for C, 47,583 for 5mC, 35,976 for 5hmC, 43,155 for 5gmC and 44,223 for N₃-5gmC, suggesting that the labeling does not have a significant impact on sequencing yield.

As shown in FIG. 1A, 5-methylcytosine exhibited a clear shift in signal relative to unmodified cytosine, whereas the signal of 5-hydroxymethylcytosine was difficult to distinguish from that of either 5-methylcytosine or unmodified cytosine. However, modification with glucose or azidoglucose resulted in a pronounced shift, which clearly distinguished the signal from that of cytosine and 5-methylcytosine.

Similarly, the current distribution signals associated with adenine, methylated adenine (m6A) and azide-modified adenine (N3-A) in the sequence GATCGAATATT (SEQ ID No. 8) were compared. This motif was selected to contain the sequence TCGA, the recognition motif for M.TaqI methyltransferase. 35,832 reads were found for the canonical amplicon, 47,583 reads for the m6A amplicon and 44,223 reads for the azide-m6A amplicon, suggesting that the azide does not have a significant impact on sequencing yield.

As shown in FIG. 1B, the current distribution signals of adenine and m6A were highly similar, whereas the signal from azide-m6A exhibited a strong shift relative to that of unmodified adenine.

These results indicate that labeling 5hmC with glucose or azidoglucose and labeling methylated adenine with azide can be useful in identifying modified nucleotides using nanopore technology, and distinguishing them from unmodified nucleotides.

Use of Mtaq can identify unmethylated cytosine by adding a bulky group onto adenine. It may also be used for DAM-ID assays, where the DAM MTase is fused to a DNA binding protein and adds adenine where the protein is attached. It can also be used for chromatin accessibility assay where DAM labels adenines only in open chromatin regions that can be identified in the nanopore It can identify unmethylated adenines in DNA (i.e., label only unmethylated adenines). It can be used to detect RNA modifications which are very important in translation regulation.

In addition to the simple shifts in current observed in FIGS. 1A-B, four nanopore features were computationally determined: skipped events, unidentified k-mers, modulation in dwell time. and shift in current from the model k-mer.

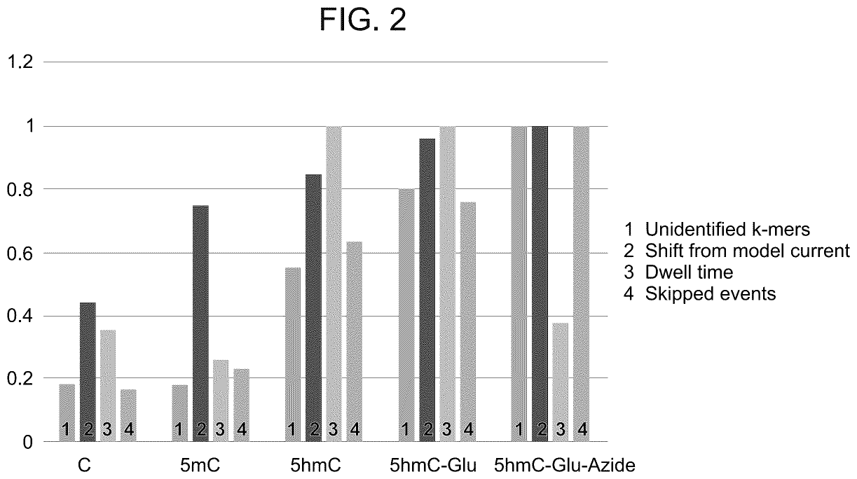

As shown in FIG. 2, the various modified cytosines and unmodified cytosine exhibited different degrees of the various nanopore parameters, indicating that these features themselves can help to enhance accuracy of base modification with even simple modeling.

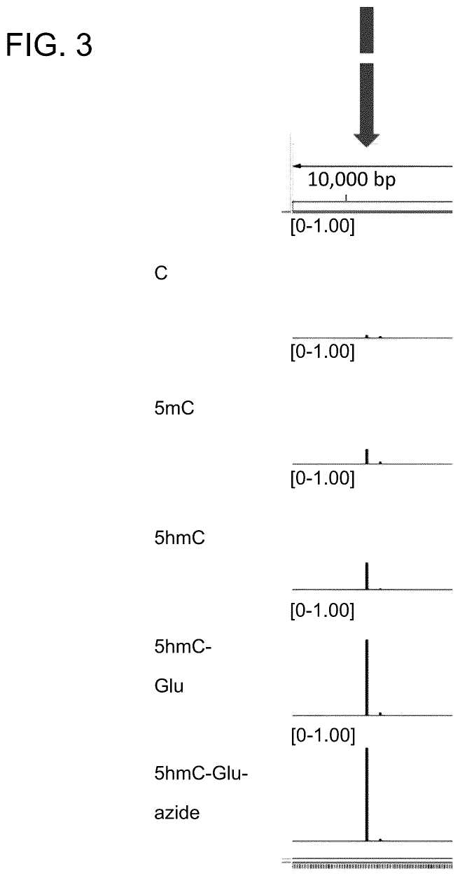

As shown in FIG. 3, labeling of 5hmC with glucose or azidoglucose enhanced the observed peak associated with modified cytosine in a genome browser view.

The ability to recognize native 5hmC in genomic DNA was then investigated. To this aim,

DNA from mouse brain (cortex) tissue was extracted, as this tissue is known to present relatively high levels of 5hmC. The 5hmC was chemically modified to generate 5hmC-Glu-azide groups, and the DNA was subjected to library construction and nanopore sequencing, using procedures such as described hereinabove. Results were analyzed according to current modulations and four nanopore parameters, followed by filtering out the lowest 10% signal and signal that is not within CpG sites. These results were then compared to publicly available data of ox-BS-seq from mouse cortex.

As shown in FIG. 4, the peaks representing labelled 5hmC sites as identified by nanopore sequencing were similar to the 5hmC pattern in the publicly available data; whereas in the control sample containing non-labelled 5hmC sites, the number of identified 5hmC sites was considerably smaller than in the publicly available data.

These results indicate that the addition of a bulky group resulted in enhanced electrical contrast that can be utilized to provide a more accurate identification of epigenetic modifications.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1. A method of detecting a presence or absence of at least one labeled nucleobase in a polynucleotide molecule, comprising:
assaying a polynucleotide molecule using a nanopore device having an average pore diameter of less than 5 nanometer, wherein at least one labeled nucleobase in said polynucleotide molecule has a detectable moiety having a molecular weight that ranges from 40 to 1,000 Daltons attached thereto, thereby detecting a presence or absence of said at least one labeled nucleobase of said polynucleotide molecule,
wherein a presence of said at least one labeled nucleobase is indicative of an initial absence of a modified nucleobase at or near a position of the labeled nucleobase.

2. A method of detecting a presence or absence of a single naturally-or synthetically-modified nucleobase in a polynucleotide molecule, the method comprising:
(a) contacting said polynucleotide molecule with one or more reagents capable of selectively attaching a detectable moiety to at least one said naturally-or synthetically-modified nucleobase in said polynucleotide molecule or to at least one nucleobase adjacent to at least one naturally-or synthetically-modified nucleobase in said polynucleotide molecule, to form a labeled nucleobase in said polynucleotide molecule, wherein said presence or said absence of said modified nucleobase is determined by said attaching; and
(b) assaying said polynucleotide molecule using a nanopore device,
thereby detecting a presence or absence of said labeled nucleobase of said polynucleotide molecule,
wherein a presence of said labeled nucleobase is indicative of an initial absence of said modified nucleobase at or near the position of the labeled nucleobase.

3. The method of claim 2, wherein said nanopore device has an average pore diameter of no more than 5 nanometer.

4. The method of claim 2, wherein said detectable moiety has a molecular weight of from 40 to 1,000 Daltons.

5. The method of claim 2, wherein said one or more reagents comprise a methyltransferase or methylase enzyme selected from the group consisting of adenine methylase or CpG methylase, and/or DAM methyltransferase, Taq1 methyltransferase, Alu1 methyltransferase, BamH1 methyltransferase, CpG methyltransferase (M.SssI), CpG methyltransferase (M.MpeI), GpC methyltransferase (M.CviPI), EcoG2 methyltransferase, EcoRI methyltransferase, Hae3 methyltransferas, HhaI, and Hpa2 methyltransferas, and/or Msp1 methyltransferas, optionally in combination with 5′-methylthioadenosine/S-adenosylhomocysteine nucleosidase (MTAN) enzyme.

6. The method of claim 2, further comprising determining a sequence of said polynucleotide molecule by nanopore sequencing.

7. The method of claim 6, wherein said nanopore device is a protein nanopore device.

8. The method of claim 2, wherein said one or more reagents are capable of selectively attaching said detectable moiety to said modified nucleobase, and a presence of said labeled nucleobase is indicative of an initial presence of said modified nucleobase at the position of the labeled nucleobase.

9. The method of claim 2, wherein said modified nucleobase is 5-hydroxymethylcytosine.

10. The method of claim 9, wherein said one or more reagents comprise β-glucosyltransferase and a uridine diphosphoglucose that comprises a substituted or non-substituted glucose moiety.

11. (canceled)

12. The method of claim 2, wherein said one or more reagents comprise a methyltransferase or methylase, such as adenine methylase or CpG methylase and/or in combination with MTAN enzyme.

13. The method of claim 12, wherein said one or more reagents further comprise an S-alkyl-S-adenosyl-homocysteine or synthetic analog thereof.

14. The method of claim 2, wherein said modified nucleobase is a methylcytosine or a methyladenine.

15. The method of claim 14, being for detecting an absence of 5-methylcytosine in a CpG dinucleotide.

16. The method of claim 15, wherein said modified nucleobase is a modified adenine adjacent to said CpG dinucleotide.

17. The method of claim 16, wherein said one or more reagents comprise DAM methyltransferase, Taq1 methyltransferase, Alu1 methyltransferase, BamH1 methyltransferase, CpG methyltransferase (M.SssI), CpG methyltransferase (M.MpeI), GpC methyltransferase (M.CviPI), EcoG2 methyltransferase, EcoR1 methyltransferase, Hae3 methyltransferas, Hha1, Hpa2 methyltransferas, DAM MTase and/or Msp1 methyltransferas optionally in combination with MTAN enzyme.

18. The method of claim 2, further comprising analyzing data obtained from said nanopore device.

19. The method of claim 18, wherein said analyzing data comprises analyzing at least one parameter selected from the group consisting of shift in current, skipped events, unidentified k-mers, and modulation in dwell time.

20. The method of claim 2, wherein a shift in current of said labeled nucleobase relative to a corresponding standard nucleobase is greater than a shift in current of said modified nucleobase relative to a corresponding standard nucleobase.

21. The method of claim 20, wherein said shift in current of said labeled nucleobase is at least two standard deviations greater or smaller than said shift in current of said modified nucleobase.
